(19) United States
(12) Patent Application Publication
(10) Pub. No.: US 2025/0161389 A1
(43) Pub. Date: May 22, 2025

(54) Herbal composition for the treatment of malaria

(71) Applicants: Matthias Magoola, Kampala (UG); Sarfaraz K. Niazi, Deerfield, IL (US)

(72) Inventors: Matthias Magoola, Kampala (UG); Sarfaraz K. Niazi, Deerfield, IL (US)

(73) Assignee: RNA Therapeutics, Inc., Deerfield, IL (US)

(21) Appl. No.: 18/513,584

(22) Filed: Nov. 19, 2023

(51) Int. Cl. A61K 36/24; A61K 31/122; A61K 31/155; A61K 31/357; A61K 31/47; A61K 31/4706; A61K 31/4709; A61K 31/65; A61K 36/185; A61K 36/19; A61K 36/48; A61K 36/736; A61K 36/758; A61P 33/06

(52) U.S. Cl. CPC A61K 36/24; A61K 31/122; A61K 31/155; A61K 31/357; A61K 31/47; A61K 31/4706; A61K 31/4709; A61K 31/65; A61K 36/185; A61K 36/19; A61K 36/48; A61K 36/736; A61K 36/758; A61P 33/06; A61K 2236/333

(57) ABSTRACT

An herbal composition comprising Harungana madagascariensis, Carissa edulis, Justicia betonica, Alstonia boonei, Zanthoxylum gillettii, Prunus africana, Newtonia buchananii, and combinations thereof for treating malaria.

### SUMMARY OF THE INVENTION

Malaria remains a dreadful disease despite many advances in drug development and other modalities for its prevention. To thwart the death of about half a million patients from malaria, the attention now draws to evaluating anecdotal medicines that have been used for centuries. Throughout history, anecdotal medicines, often based on traditional knowledge and empirical observations, have played a significant role in healthcare. These remedies, passed down through generations, were initially regarded with skepticism by the medical establishment. However, over time, some of these anecdotal medicines have proven remarkably effective and contributed to the development of modern medicine. Examples include the use of willow bark for pain relief (which led to the discovery of aspirin), indigenous plant-based remedies for various ailments, and the therapeutic properties of certain foods. While anecdotal evidence alone is insufficient for establishing medical efficacy, it has often served as a valuable starting point for scientific investigation, leading to the development of evidence-based medicine and the validation of many traditional remedies.

This invention presents a most effective treatment of malaria that involves using seven herbs: Harungana madagascariensis, Carissa edulis, Justicia betonica, Alstonia boonei, Zanthoxylum gillettii, Prunus africana, Newtonia buchananii, administered as dried powder or as powdered hydro-alcoholic extra in a small dose of 3 g twice a day for three days. To date, these herbs have not been reported to have the claim of effectiveness in the mode of administration of this invention.

### BACKGROUND OF THE INVENTION

Malaria is a pervasive and deadly infectious disease caused by Plasmodium parasites and transmitted through the bite of infected female Anopheles mosquitoes. Its impact varies by region, with the most significant global burden in tropical and subtropical areas. In 2020, there were approximately 229 million malaria cases globally, leading to around 409,000 deaths, with the majority occurring in sub-Saharan Africa. Vulnerable populations, particularly children under five and pregnant women, bear the brunt of this disease. Over the past two decades, progress has been made in reducing malaria cases and deaths through initiatives such as insecticide-treated bed nets, indoor spraying, and effective antimalarial drugs. Nevertheless, challenges persist, including drug and insecticide resistance, healthcare system limitations, and the need for sustained global efforts, research, and innovation to combat malaria and work toward its elimination. Various organizations and global initiatives are dedicated to this ongoing battle, aiming to reduce the malaria burden and eventually achieve a world free of this deadly disease.

Efforts to genetically modify mosquitoes responsible for malaria transmission represent innovative strategies in the fight against the disease. These initiatives mainly target Anopheles mosquitoes, the primary vectors of Plasmodium parasites causing malaria. Gene drive technology has been developed to bias the inheritance of specific genes, potentially reducing mosquito populations by spreading genes, rendering them incapable of transmitting malaria, or limiting their reproduction. Researchers have also explored genetic modifications within mosquitoes, aiming to block the development of malaria parasites. This includes genetically modified mosquitoes producing antibodies against Plasmodium, inhibiting parasite growth. The Sterile Insect Technique (SIT) involves breeding sterilized male mosquitoes, using genetic modification to induce sterility.

Additionally, introducing Wolbachia bacteria into mosquito populations has shown promise in reducing malaria transmission. Ethical, environmental, and safety considerations are crucial in these genetic modification efforts, complementing malaria control measures like bed nets and antimalarial drugs. While these strategies hold potential, they are still undergoing research, regulatory scrutiny, and ecological assessments before widespread implementation.

The treatment of malaria in African countries involves a multifaceted approach aimed at reducing its impact. Primary treatment relies on antimalarial drugs, particularly Artemisinin-Based Combination Therapies (ACTs), which are highly effective against the most dangerous malaria parasite, Plasmodium falciparum. Timely and accurate diagnosis, often facilitated by rapid diagnostic tests (RDTs) or microscopy, is crucial. Access to medicines is improved through initiatives like the Global Fund, which provides funding to enhance drug availability. Preventive measures, including distributing insecticide-treated bed nets (ITNs) and indoor residual spraying (IRS), are vital in curbing transmission. Community health workers are pivotal in diagnosing and treating uncomplicated cases, especially in remote areas. Public awareness campaigns and education programs inform communities about the importance of early treatment and preventive measures. Challenges such as limited healthcare infrastructure and drug resistance persist, but international organizations like WHO, governments, and NGOs collaborate to strengthen healthcare systems and combat malaria. Research and development efforts continue to improve treatment options, develop new drugs, and work toward malaria eradication. Overall, a comprehensive strategy is essential to control and treat malaria effectively in African countries.

Antimalarial drugs can become ineffective over time due to the development of drug-resistant strains of the malaria parasite Plasmodium. Malaria parasites have a remarkable ability to adapt and evolve, which can lead to resistance to the drugs used for treatment. Resistance typically emerges when parasites that carry genetic mutations allowing them to survive drug exposure are selected for and become more prevalent in a population. This natural selection process occurs when antimalarial drugs are widely and indiscriminately used, especially when not taken in the correct doses or treatment is not completed. As a result, previously effective drugs may lose their effectiveness against certain strains of malaria, posing a significant challenge to malaria control efforts. To address this issue, it is crucial to continually monitor drug resistance, develop new antimalarial drugs, and implement strategies that promote the rational use of existing drugs to slow down the development of resistance and ensure effective treatment.

The history of herbal medicine in Africa is a testament to ancient healing practices that have thrived for millennia. Indigenous African cultures, including those in Egypt, Nubia, and Ethiopia, developed an extensive knowledge of medicinal plants, documenting their use on papyrus scrolls and inscriptions. This tradition of using herbs for healing was passed down through generations, with traditional healers or herbalists playing a vital role in preserving and transmitting this knowledge. Herbal medicine addresses various ailments, from fevers and infections to digestive disorders and wounds. Importantly, it was not just about physical healing; it held spiritual and cultural significance, often involving rituals to promote well-being and ward off evil forces. Despite colonial challenges, traditional healing systems persisted. Today, there is a growing recognition of the value of African herbal medicine within national healthcare systems, alongside efforts to conserve and sustainably harvest medicinal plants. African herbal medicine remains a bridge between traditional wisdom and modern healthcare, offering insights into the healing power of nature.

Despite the advances made in the accessibility to drugs to treat malaria, there remains a dire need for an effective, safe, affordable, and readily available drug: a proprietary combination of seven herbs, including Harungana madagascariensis, Carissa edulis bark, Justicia betonica leaf, Alstonia boonei bark, Zanthoxylum gillettii bark, Prunus africana bark, and Newtonia buchananii bark is described in a proprietary combination and dosing regimen to treat malaria effectively.

### DESCRIPTION OF FIGURES

FIG. 1. Illustrates Anti-plasmodium activity of Harungana (h), madagascariensis Alstonia boonei (i); Zanthoxylum gilletii (j); Justicia betonica (k); Prunus africana (l); Newtonia buchananii (m); Carissa edulis (n), and a combination of (h) to (n) as (c).

### DETAILS OF THE INVENTION

Herbal medicines used to treat malaria have been employed for centuries in various traditional healing systems worldwide. While they may not be as widely recognized or extensively studied as pharmaceutical antimalarial drugs, they are crucial in some regions where access to modern medicine is limited. Here's a description of herbal medicines commonly used to treat malaria:

Artemisia annua (Sweet Wormwood): Artemisia annua, also known as sweet wormwood or Qinghao, is a well-known herb in traditional Chinese medicine. It contains the active compound artemisinin, which is highly effective against malaria parasites. Artemisinin-based combination therapies (ACTs) are now widely used as the standard treatment for malaria, and artemisinin is often combined with other herbs to enhance its antimalarial effects.

Cinchona (Quinine Tree): The bark of the cinchona tree has been historically used to treat malaria. Quinine, an alkaloid found in cinchona bark, is known for its antimalarial properties. It has been used to make quinine-based medications, but due to the development of drug-resistant malaria strains, quinine is often combined with other herbs or drugs.

Neem (Azadirachta indica): Neem leaves and extracts have been used in traditional Indian medicine (Ayurveda) to treat malaria and other ailments. Neem contains compounds like quercetin and nimbin, which have demonstrated antimalarial activity. It is used as a preventive measure and remedy for malaria symptoms.

African Wormwood (Artemisia afra): African wormwood is another species of the Artemisia genus with potential antimalarial properties. It has been used in traditional African medicine to treat fever and malaria. Like Artemisia annua, it contains compounds that may help combat malaria parasites.

Papaya (Carica papaya): Papaya leaf extract is a traditional remedy for malaria in some tropical regions. It is believed to boost the body's immune system and help recover from malaria. While not a direct antimalarial treatment, it may alleviate some symptoms and support overall health during recovery.

Ginger (Zingiber officinale): Ginger is known for its anti-inflammatory and immune-boosting properties. In some cultures, ginger is an adjunct therapy to alleviate fever and nausea associated with malaria.

Tinospora cordifolia (Giloy): Giloy, an herb used in traditional Ayurvedic medicine, is believed to enhance the body's immunity and may have antimalarial effects. It is sometimes used to support recovery from malaria.

In the ongoing battle against malaria, a synergy between modern medicine and traditional herbal remedies is increasingly recognized as a valuable approach. While pharmaceutical antimalarial drugs like artemisinin-based combination therapies (ACTs) remain the gold standard for treatment due to their proven efficacy and controlled dosages, herbal medicines continue to be explored and studied for their potential benefits. Researchers are actively investigating the active compounds in plants like Artemisia annua and exploring how they can be harnessed to develop more effective antimalarial treatments. Moreover, traditional herbal knowledge is preserved and passed down through generations, contributing to a holistic approach to healthcare in regions where malaria remains a significant health concern. Integrating traditional wisdom with modern scientific research can lead to a more comprehensive and effective strategy for malaria prevention and treatment, ultimately bringing us closer to the goal of eradicating this devastating disease.

The ongoing exploration and integration of traditional herbal remedies into modern healthcare practices reflect the evolving understanding of how different cultures and approaches can contribute to the global fight against malaria. Collaboration between traditional healers, scientists, and healthcare professionals is essential in this effort, as it allows for the exchange of knowledge and the development of evidence-based treatments. Furthermore, as drug-resistant malaria strains continue to emerge, the diverse array of potential antimalarial compounds found in various plants may hold promise for developing new medications. However, rigorous scientific research, clinical trials, and safety assessments are necessary to validate the efficacy and safety of herbal medicines and their integration into mainstream malaria control programs. By combining the strengths of traditional wisdom and modern scientific methodologies, we can continue to progress in the battle against malaria, aiming for a future where this disease becomes a thing of the past.

The present invention is based on a proprietary combination of seven herbs, administered equally in a total dose of 3 g of powdered part of the plant or its equivalent extract administered twice daily for three days to treat malaria. The components of the combination include Harungana madagascariensis, Carissa edulis bark, Justicia betonica leaf, Alstonia boonei, Zanthoxylum gillettii bark, Prunus africana bark, and Newtonia buchananii bark. The active ingredients in herbs can vary widely and may include a range of compounds with potential medicinal properties. Any part of the plant, bark, root, leaf, or flower can contain antimalarial activity.

Pharmacological assessment of the antiprotozoal activity, cytotoxicity, and genotoxicity of medicinal plants used to treat malaria in the Greater Mpigi Region of Uganda have been reported. Still, they do not identify the herbs claimed in the present invention and their proof of activity. [Attachment 1: Schultz F, Osuji O F, Nguyen A, Anywar G, Scheel J R, Caljon G, et al. Pharmacological Assessment of the Antiprotozoal Activity, Cytotoxicity and Genotoxicity of Medicinal Plants Used to Treat Malaria in the Greater Mpigi Region in Uganda. Frontiers in Pharmacology. 2021;12.]

Another study [Attachment 2: Lemma M T et al. J. Medicinal plants for in vitro antiplasmodial activities: A systematic literature review. Parasitol Int. 2017 December;66(6):713-720.], the medicinal plants with very good in vitro antiplasmodial activities, with half-maximal inhibitory concentration (IC50)≤1 μg/ml, and to determine trends in the process of screening their antiplasmodial activities. A systematic analysis of 58 reports categorized them as very good, good, moderate, and inactive if the IC50 values were <0.1 μg/ml, 0.1-1 μg/ml, >1-5 μg/ml, and >5 μg/ml respectively. This study documented 752 medicinal plants belonging to 254 genera. Most of the plants were reported from Africa, followed by Asia. About 80% of the plants experimented were reported to be inactive. Among plants with very good anti-plasmodial crude extracts are Harungana madagascariensis, Quassia africana, and Brucea javanica. At the same time, Picrolemma spruce, Aspidosperma vargasi, Aspidosperma desmanthum, and Artemisia annua were reported to have individual compound isolates with very good antiplasmodial activities.

In another recent study, a systematic review of 150 plant species was screened in vitro, one in vivo and 46 in vivo and in vitro. Three hundred and forty-four of the tests reported good activity (IC50<10 μg/mL or parasite suppression rate of ≥50%), 414 moderate activity (IC50 values of 10-49 μg/mL or parasite suppression rate of 30%-49%) and 412 were reports of inactivity (IC50>50 μg/mL or parasite suppression rate of <30%). Fuerstia africana and Ludwigia erecta were reported to have the highest activities, with IC50<1 μg/mL against Plasmodium falciparum D6 strain and chemosuppression in mice at an oral dose of 100 mg/kg, was reported as 61.9% and 65.3% respectively. Fifty-five antimalarial/antiplasmodial active compounds isolated from eight plant species were reported, with resinone (39) having the best activity (IC50<1 μg/mL). Of the 197 plant species, the most studied plant families were Asteraceae, 16 (8.1%), Verbenaceae, 9 (4.6%), Rubiaceae, 8 (4%), Fabaceae, 7 (3.6%) and Leguminosae, 7 (3.6%). The most investigated plant species were Rotheca myricoides (Hochst.) Steane and Mabb Azadirachta indica A. Juss., Rhus natalensis Bernh. ex Krauss, Turraea robusta (Hochst.) Benth., Ximenia americana L., Vernonia auriculifera Hiern, Toddalia asiatica (L.) Lam., Maytenus undata (Thunb.) Blakelock, Lannea schweinfurthii (Engl.) Engl., Zanthoxylum chalybeum Engl., Harrisonia abyssinica Oliv. Fuerstia africana Oliv. and Asparagus racemosus Willd. Leaves, 85 (27%); stem barks, 87 (28%); root barks, 83 (26%) and whole plant 28 (9%) were the most common parts of the plants used to prepare extracts). Crude extracts dominated in the tests compared to tests done using isolated compounds at 1072 (91.6%) and 98 (8.4%), respectively. Moreover, most extracts were organic 401 (67.3%) compared to aqueous extracts 195 (32.7%). In ascending order: 1:1 mixture dichloromethane:methanol, 9 (1.5%), hexane, 13 (2.2%), chloroform, 13 (2.2%), petroleum ether, 17 (2.9%), ethyl acetate, 18 (3%), dichloromethane, 19 (3.2%), water, 195 (32.7%) and methanol 309 (51.8%) were the most frequent extraction solvents used. [Attachment 3: Irungu, B., et al., Potential of medicinal plants as antimalarial agents: a review of work done at Kenya Medical Research Institute. Front Pharmacol, 2023. 14: p. 1268924.]

The herbs that are claimed in the present invention were assigned a code for blinding purposes, which are shown below in parentheses after their name:

Harungana madagascariensis (h) (Hypericaceae family) thrives in tropical and subtropical climates and can be found in various habitats, from lowland forests to savannas. Despite its potential benefits and cultural significance, the plant may face habitat loss and deforestation threats in some regions. While it is not currently listed as a threatened or endangered species, conservation efforts to protect its natural habitat may be essential to ensure its continued existence. When considering the use of this plant for medicinal purposes or traditional practices, it is crucial to consult with healthcare professionals or experts in traditional medicine to ensure safety and efficacy and to respect local cultural practices and conservation concerns. Harungana madagascariensis is widely used in traditional African medicine. This plant's leaves, bark, and roots are utilized to treat various health conditions. The leaves are often applied topically to soothe skin irritations, such as rashes and wounds. Infusions or decoctions made from the bark or roots have been employed as remedies for stomachaches, diarrhea, and respiratory infections.

Additionally, Harungana madagascariensis has shown promise as an antimalarial agent due to its antiplasmodial properties, making it a potential resource in the fight against malaria. Its active ingredient may include Harunganin, a compound with potential anti-inflammatory properties in the bark, and quinones that may contribute to its medicinal properties. This plant has been used to treat several diseases, such as jaundice, typhoid fever, anemia, and skin and heart problems. They correlate with different pharmacological tests involved with different plant extracts and can be identified as antioxidants and antitrichomonal.

Alstonia boonei (i) (Apocynaceae family), commonly known as the African rubber tree or the Alstonia tree, is a species of evergreen tree native to tropical and subtropical regions of Africa. Its tree is known for its tall stature, often reaching heights up to 30 meters (approximately 100 feet). Alstonia boonei has a smooth, grayish bark and glossy, dark green leaves arranged in whorls. It produces clusters of small, white, fragrant flowers, followed by elongated, slender seedpods. While it is primarily recognized as a valuable source of latex used in traditional African medicine and local industries, various parts of the tree, including the bark, leaves, and roots, have been used for their medicinal properties in treating various ailments such as malaria, fever, and gastrointestinal issues. The wood from Alstonia boonei is valued for its timber, and the tree has cultural significance in some African communities. Alstonia boonei, the African rubber tree, is significant in traditional African medicine. The tree's bark is the most commonly used part to treat various health issues, including malaria, fever, and gastrointestinal disorders. Its bitter compounds are believed to have antipyretic (fever-reducing) and antimalarial properties. In some cultures, a decoction of the bark is taken orally or used as a topical application for various ailments. Its active ingredient may include alkaloids in the bark, such as echitamine and strictamine, triterpenoids, and alkaloids, with anticipated therapeutic effects.

Zanthoxylum gillettii (j) (Rutaceae family), commonly known as Gillett's prickly ash or African pepper, is a plant species native to tropical and subtropical regions of Africa, including countries like Nigeria, Cameroon, and Uganda. It is known for its distinctive features, including compound leaves with numerous small leaflets and sharp thorns on its branches. The plant's aromatic and pungent seeds, bark, and leaves are used for their culinary and medicinal properties in various African cuisines and traditional medicines. In cooking, the seeds are employed as a spice and flavoring agent, imparting a citrusy and spicy taste to dishes. Medicinally, Zanthoxylum gillettii has been utilized to treat various ailments, including digestive issues and toothaches, and as a general tonic. Its active compounds, such as alkaloids and essential oils, contribute to its medicinal and culinary applications, making it a valuable plant with a long history of use in African cultures. Zanthoxylum gillettii, or Gillett's prickly ash, has culinary and medicinal applications. African cuisines use its seeds, bark, and leaves as a spice and flavoring agent due to their citrusy and spicy flavor. Medicinally, it has been used for digestive complaints, including indigestion and stomachaches. It has been applied topically to soothe toothaches. The plant's essential oils and alkaloids contribute to its potential therapeutic benefits. Zanthoxylum gillettii, or Gillett's prickly ash, has culinary and medicinal applications. African cuisines use its seeds, bark, and leaves as a spice and flavoring agent due to their citrusy and spicy flavor. Medicinally, it has been used for digestive complaints, including indigestion and stomachaches. The plant's essential oils and alkaloids contribute to its potential therapeutic benefits. Its active ingredients may include alkaloids, including compounds like berberine and canthin-6-one, and the plant's essential oils may contain bioactive compounds with potential health benefits.

Justicia betonica (k) (Acanthaceae species) is a flowering plant species. It goes by the common name squirrel's tail and paper plume, commonly known as the white shrimp plant, and is a flowering species. Native to tropical regions of Central and South America, including countries like Brazil, Peru, and Ecuador, this perennial herbaceous plant is known for its striking and distinctive white bracts that resemble shrimp. It typically reaches a height of around 3 to 4 feet (approximately 1 to 1.2 meters) and produces vibrant tubular flowers that emerge from these white bracts. Justicia betonica is favored in ornamental gardening for its unusual and eye-catching appearance, making it a popular choice for gardens and landscapes. Beyond its ornamental value, it has limited documented traditional medicinal uses in some regions but is primarily cultivated for its aesthetic appeal and unique floral display. Justicia betonica, though primarily valued for its ornamental qualities, has limited traditional medicinal uses in some regions. It has been used in folk medicine for its potential to address minor ailments, such as mild digestive issues, or as a gentle herbal remedy. However, its medicinal applications are less well-documented or researched than other plants. It is primarily valued for its ornamental qualities and may have limited documented therapeutic compounds.

Prunus africana (1) (Rosaceae family), commonly known as African cherry, pygeum, or African plum, is a tree species native to the highland regions of Central and East Africa, including countries like Cameroon, Kenya, and Madagascar. This evergreen tree can reach up to 40 meters (approximately 130 feet) and is recognized for its dark, fissured bark and glossy, elliptical leaves. Prunus africana is particularly renowned for its medicinal properties, as its bark has been traditionally used in African and traditional herbal medicine for its potential to treat various ailments. It is most notably used to alleviate symptoms of benign prostatic hyperplasia (BPH), a common condition in older men characterized by an enlarged prostate gland. The bark contains bioactive compounds, including phytosterols and pentacyclic triterpenes, which are believed to contribute to its therapeutic effects. Due to its medicinal importance and overharvesting concerns, Prunus africana has been the subject of conservation efforts to ensure its sustainability. Prunus africana, or African cherry, is primarily known for its bark, which has been extensively used in traditional medicine. It is especially recognized for its potential to alleviate symptoms of benign prostatic hyperplasia (BPH) in men. Extracts from the bark contain phytosterols and pentacyclic triterpenes, which are believed to have anti-inflammatory and antiandrogenic effects, making them helpful in managing prostate-related conditions. The bark of Prunus africana contains phytosterols, such as beta-sitosterol, which are believed to have anti-inflammatory and other therapeutic effects. It also contains pentacyclic triterpenes, including ursolic acid and oleanolic acid, and alkaloids that are thought to contribute to the plant's medicinal properties.

Newtonia buchananii (m) (Fabaceae family), commonly known as Buchanan's newtonia or forest newtonia, is a tree species native to tropical and subtropical regions of Africa, primarily found in countries such as Ghana, Nigeria, and Cameroon. Its tree typically reaches up to 30 meters (approximately 100 feet) and has a distinctive, spreading crown with pinnately compound leaves. The tree produces small, fragrant, cream-colored flowers that pollinators like bees visit. Newtonia buchananii is ecologically important as it provides habitat and food for various wildlife species. In addition to its ecological significance, Newtonia buchananii has cultural and economic importance in many African communities. The tree's wood is highly valued for its durability and is used in construction, carpentry, and for making traditional instruments. The tree leaves and bark have been used in traditional medicine for various purposes, including treating malaria, diarrhea, and skin conditions. While more research is needed to validate its medicinal properties, Newtonia buchananii plays a vital role in the natural environment and the cultural heritage of the regions where it is found. Conservation efforts are also important to ensure the sustainability of this valuable tree species. It has been utilized in traditional medicine in some African communities. The tree's leaves and bark are used for various purposes, such as treating gastrointestinal issues and as a general tonic. However, it's important to note that this plant's specific medicinal properties and active compounds are not as extensively studied or documented as other medicinal plants.

Carissa edulis (n) (Apocynaceae family), the Natal plum, is found in many communities worldwide. It has been traditionally used to treat conditions such as headaches, chest complaints, rheumatism, and sexually transmitted infections like gonorrhea and syphilis, rabies, herpes, and malaria. It is also known for its diuretic effects. Carissa edulis also manages oxidative stress and inflammation-related disorders, including malaria, rheumatic inflammation, and cardiovascular diseases. In Kenya, for example, the plant is used widely in traditional medicine for various ailments and is noted for not having reported side effects. The roots have been used for their antiherpetic properties. Moreover, the plant has been shown to regulate vascular tone and has antioxidant and anti-inflammatory properties. Specific studies have demonstrated that extracts from Carissa edulis root bark can inhibit the activity of cyclooxygenase-2 (COX-2), an enzyme involved in inflammation and pain.

### Experimental Details

Specifically, experiments were designed to (a) prepare standard extracts containing bioactive polar and non-polar secondary metabolites from dried plants, (b) screen the extracted materials for their antimalarial activity by measuring inhibitory effects on the growth of Plasmodium falciparum in the laboratory, and (c) fractionate, isolate and purify potential antimalarial compounds. To this effect, they were supplied for extraction using either water or methanol as extracting solvents, and then each of the extracts was evaluated for potential antimalarial activity. All plant samples were dried and pulverized powders whose identity (e.g., species, parts collected, voucher specimens, etc.) were coded and not disclosed to the testing team. The samples were coded form “h” to “n” for Harungana madagascariensis, Carissa edulis, Justicia betonica, Alstonia boonei, Zanthoxylum gillettii, Prunus africana, Newtonia buchananii, respectively.

The samples were extracted using methanol and water; the resultant extracts were then tested for their in vitro activity against Plasmodium falciparum. Initially, in vitro antimalarial activity screening was conducted on crude extracts, where plants were evaluated for dose-dependent inhibition of the growth of two strains of P. falciparum parasites. Comparisons on the potency and efficacy of these compounds were made based on the minimum inhibitory concentrations of each plant extract. For extraction, two solvents, (a) alcohol (methanol) and (b) water, were used to extract bioactive ingredients. The antimalarial activity was determined by either SYBR Green I or HRP-ELISA assays, as described below.

Solvent extraction, liquid-liquid partitioning, and TLC analysis of each sample were performed to determine the expected percent yield and the most suitable starting solvents for purification. Here, the dried and pulverized plant materials were initially air dried in an incubator at constant temperature (at 37° C. for 15 minutes) to remove residual moisture. About 100 g of each sample provided was extracted with either methanol or deionized distilled water (ddH2O) to prepare extracts. Briefly, water (H2O) extracts were prepared by weighing 100 g of the dried powder and soaking it in 200 mL of pre-warmed de-ionized distilled water (40-50° C.) for 3 h, and then left to stand at room temperature overnight. In the second extraction procedure, 100 g of the dried powder was soaked in 200 mL of 95% aqueous methanol (analytical grade) and stored in the dark for 24 h with intermittent shaking to prepare methanolic extracts. In either method, crude extracts were filtered using 70 mm diameter Whatman filter paper (Grade 1 and concentrated using a rotary evaporator under reduced pressure. The residual solvent (mainly water) was removed by drying overnight using a Speed Vac equipped with a refrigerated vapor trap. These extraction steps afforded brownish to green powder extractives, and these were kept in air-tight bottles and stored at −20° C. in the freezer until used in subsequent experiments.

After the initial pilot extraction step, the extraction was scaled up (10-fold) to obtain enough samples for fractionation, isolation, and identification of compounds. About 1 kg of plant material was soaked in 5 liters (1:5) of high-grade methanol (HPLC grade) in a dark bottle. The soaked material and solvent were left to extract in the dark for 24 hours with intermittent vigorous shaking. Methanol is a general solvent that breaks the cell walls of plant cells to liberate various compounds, including secondary metabolites. To ensure exhaustive extraction, the extraction procedure was repeated about three times with fresh solvent each time until the solvent became near colorless, indicating complete extraction. These crude methanolic extracts were double-filtered and concentrated at reduced pressure by vacuum rotary evaporation. The residual solvent was removed by drying samples in a Speed Vac with an attached refrigerated vapor trap.

Fractionation of the extracts was done by liquid-liquid partitioning and preparative HPLC methods. In the first step of fractionation (defatting), the dried methanol extracts were weighed and resuspended in 100 ml of aqueous methanol (90%). Defatting was done by partitioning the extracts dissolved in methanol with an equal volume of n-hexane (100 ml). This step removed the highly hydrophobic compounds such as chlorophylls, oils, waxes, and resins. The two fractions were separated using a separating funnel, and the two portions were each concentrated using rotary evaporation. The two fractions yielded, i.e., the methanolic and hexane fractions, were concentrated using a vacuum rotary evaporation and then dried using a speed vac at low temperature. In most samples, the hexane fractions did not dry entirely to powder but only yielded oily viscous resins. The hexane fractions were not analyzed in the subsequent samples but were stored in airtight containers at −80° C. On the other hand, the methanolic fractions were dried completely, and a brownish powder for each sample extracted was obtained.

In the next step, each defatted methanolic extract was further fractionated by solvent-solvent extraction to obtain smaller fractions with varying degrees of polarity (i.e., from the highly polar to non-polar fractions). About 20 g of defatted methanolic extracts were briefly suspended in 100 mL of deionized distilled water (5% methanol) and mixed by stirring (using a magnetic probe). Then, using a separating funnel, the extracts were partitioned sequentially with an equal volume of Chloroform, followed by Ethyl Acetate and n-Butanol. At each solvent partitioning step, the procedure was repeated three times to ensure near-complete partitioning of compounds according to polarity. All four fractions, chloroformic (C), ethyl acetate (E), butanol (B), and water (W), were concentrated by rotary evaporation and dried under vacuum to obtain powders. The composition of each fraction was monitored by analytical TLC and HPLC analysis, and the fractions that contained similar compound profiles were combined. Interestingly, water fractions from almost all samples were viscous gums that did not dry completely and were all removed from subsequent analyses, although stored at −80° C. if needed. The C, E, and B obtained from the solvent-solvent extraction step were tested for anti-plasmodial activity, and the most active fractions were selected for bio-assay guided isolation and purification of active compounds.

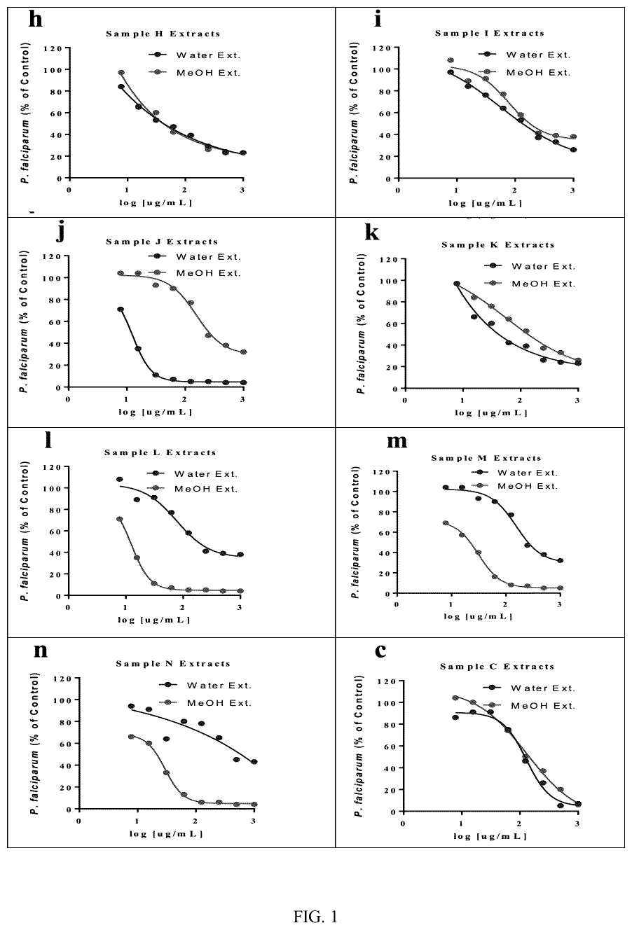

The crude fractions were further fractionated for each sample over a C₁₈ reverse phase resin column, using gradient mixtures of acetonitrile-water (10%→100%), affording 10 fractions. Briefly, extracts were dissolved in aqueous methanol, loaded onto the column, and allowed to adsorb to the column for 1-2 h. The compounds were eluted from the column using increasing acetonitrile concentrations in water. The 10 fractions were dried and analyzed by analytical HPLC to determine compound profiles and identify target compounds for isolation by preparative HPLC. Aliquots from each fraction were prepared and dried using a Speed Vac. These aliquots were chromatographed using preparative HPLC on RP18-HPLC using the following parameters: stationary phase: Reverse Phase C₁₈ column 80 g, 40 μm; mobile phase: A=H₂O, B=acetonitrile; gradient mixtures: are indicated in the isolation chromatograms for each sample; flow rate 20 mL/min; solid sample injection: trituration of the sample with loose RP18 material (1.5 g) filled in a 75 mL dry load cell; detection: ELSD, UV (254, 280 nm); collection mode: collect all; tube volume: 5 mL (peak), 20 mL (nonpeak); UV and ELSD sensitivity: low. The isolated compounds were each monitored by analytical HPLC and dried under vacuum pump evaporation. The compounds were sent for structural elucidation, NMR, LCMS/MS, and Mnova processing. (FIG. 1)

### Antimalarial Assays

The anti-plasmodial activity as a measure of antimalarial efficacy was performed using standard in vitro anti-plasmodial assays, SYBR Green I, and HRP2 ELISA assays. In this study, the Plasmodium falciparum strains 3D7 (known to be CQ sensitive) and Dd2 (known to be CQ resistant) were used to screen for antimalarial activity. First, stock concentrations of extracts (10 mg/ml) were prepared in DMSO, centrifuged to remove insoluble materials, and stored at −80° C. until used in cell culture. Before incubation with plasmodium, DMSO stocks extracted were diluted (as indicated in each assay) with complete RPMI media (RPMI 1640 medium supplemented with 25 mM HEPES, 0.2% NaHCO3, 0.1 mM hypoxanthine, 100 μg/ml gentamicin, and 0.5% Albumax I [Invitrogen]). These working stocks of extracts were serially diluted 2-fold or 3-fold in a 96-well assay plate format to obtain 8-10 serial dilutions. In each assay, the final concentration of DMSO in complete media was 0.4% in a final volume of 50 ul of entire media. Drug free wells, uninfected RBC, and blank wells were used as controls for each assay plate. Dihydroartemisinin (DHA) and CQ were also serially diluted and incubated with parasites for positive control drugs on each assay. Cultures were synchronized by double sorbitol treatment and the staging and parasitemia. The parasitized whole blood samples were washed 3 times with RPMI (w/o Albumax) media at 37° C. and then resuspended in fresh RPMI media to a final hematocrit of 2%. About 150 μl of the parasite culture was added to each well into the assay plate for final parameters of 0.2% parasitemia and 2% hematocrit. Plates were incubated for 48 h or 72 hours, as indicated in each experiment, in a humidified incubator under a blood gas mixture (97% N2, 3% CO₂, 2% O₂) at 37° C. After incubation for the required time, the plates were removed and frozen. Thawed plates were processed with Sybr Green lysis buffer for one hour in the dark. The Fluorescence Intensity (FI) as a measure of parasite growth was determined using a BMG Fluostar Optima plate reader at ex 485/em 530 nm. The Fluorescence Intensity (F.I) data was plotted using GraphPad Prism 7. The IC50 values for each sample were obtained by curve fitting the data with a variable slope function. For each assay plate, a Z′ factor to assess assay quality was calculated from positive controls (drug-free wells) and negative controls (uninfected red cell control wells). The Z′ values over 0.5 were considered suitable, while assays with Z′ below 0.5 were discarded.

In other experiments (where indicated), quantification of total parasite growth was done using an enzyme-linked immunosorbent assay (ELISA) that quantitates the parasite histidine-rich protein-2 (HRP-2). Briefly, ELISA plates were pre-coated with mouse anti-HRP-2 primary/capture antibody and stored at 4-8° C. overnight. The next day, stored culture samples were diluted [1:5] in ultrapure water, hemolyzed, and 100 μL of each hemolyzed sample was added to the pre-coated ELISA plates. The sample-antibody mixture in ELISA plates was incubated at room temperature for 1 hr. Unbound or excess protein lysates were washed off three times using a wash buffer. In the next step, 100 μL of [horseradish peroxidase-conjugated] anti-mouse IgG secondary antibody was added to each well and incubated for one hour at room temperature. This was followed by four washing cycles, and 100 μL of 3,3′,5,5′-tetramethylbenzidine single-solution chromogen was added in the dark for 10 min. 50 μL of 1 M sulfuric acid was added to stop the reactions. The absorbance of each well was measured using a spectrophotometer, and the optical density [OD] values were used to measure parasite growth. The extract-free wells [drug-free] without test agents were considered 100% parasite growth and used to calculate the percent growth in other wells.

Firstly, these extracts were incubated with P. falciparum with the highest 25 mg/ml concentration and serially diluted 8 times. This was done to screen out the active and inactive samples. Interestingly, all crude extracts significantly decreased the parasite growth at these concentrations, and the dose response was not sigmoid. Therefore, it was decided that the experiment be repeated with lower concentrations, with a starting (or highest) concentration of 1 mg/ml. The water and methanolic extracts were tested against P. falciparum in the second experiment. FIG. 1 shows the dose-response data indicating each sample's activity (water and methanolic extracts).

In FIG. 1, the most active methanolic extracts were those obtained from samples l, m, and n and displayed sigmoid dose-dependent curves with efficacy of greater than 90% inhibition of parasite growth at 100 μg/ml. For the water extracts, sample j displayed the highest activity. Overall, methanolic extracts displayed greater efficacy than the water extracts.

The antiplasmodial activity is categorized in the literature as very good, good, moderate, and inactive if the IC50 values were <0.1 μg/ml, 0.1-1 μg/ml, >1-5 μg/ml, and >5 μg/ml respectively. In other studies, it is considered inactive if the IC50 value is <50 ug/ml. The activity order of the claimed plants was found to be: j, l>m, n>h>i. However, across-study comparisons may have limitations due to experimental design, extraction conditions, extraction medium, and the process of concentrating the extract. Of value is the comparative analysis. There was sufficient proof of antiplasmodial activity in all herbs in all cases.

In one embodiment, the present invention is a composition for the treatment of malaria comprising a combination of powdered roots, barks, or leaves of Harungana madagascariensis, Justicia betonica, Alstonia boonei, Zanthoxylum gillettii, Prunus africana, Newtonia buchananii and Carissa edulis.

In a second embodiment, the combination of Harungana madagascariensis, Justicia betonica, Alstonia boonei, Zanthoxylum gillettii, Prunus africana, Newtonia buchananii Carissa edulis presents each herb in an equal proportion by weight prepared by drying the herbs and then grinding or milling to form a homogenous mixture.

In a third embodiment, the composition is administered to adult patients needing treatment at 3 g twice daily for three days.

In a fourth embodiment, the composition is a dried hydroalcoholic extract.

In a fifth embodiment, the dried hydroalcoholic extract is administered in a dose equivalent to 3 g of composition before extracting and drying.

In a sixth embodiment, the composition may contain other inert pharmaceutical ingredients.

In a seventh embodiment, the composition is administered with other allopathic or herbal malaria treatments.

1. A composition administered to patients in need of treatment of malaria comprising a suitable dose of at least one herb selected from the group of herbs comprising Harungana madagascariensis, Carissa edulis, Justicia betonica, Alstonia boonei, Zanthoxylum gillettii, Prunus africana, and Newtonia buchananii, or a combination thereof and optionally containing other inert pharmaceutical ingredients.

2. The composition of claim 1, wherein the part of the plants is root, bark, or leaf.

3. The composition of claim 2, wherein the part of the Justicia betonica plant is leaf.

4. The composition of claim 1, wherein the said composition is administered in combination with an antimalarial drug selected from a group of antimalarial drugs comprising artemisinin-based compounds, chloroquine, quinine, mefloquine, doxycycline, atovaquone-proguanil, primaquine, and artesunate, or a combination thereof.

5. The composition of claim 1, wherein the herbs are administered as a dried powdered mixture.

6. The composition of claim 5, wherein the herbs are administered as a hydro-alcoholic extract as liquid or as dried powder.

7. The composition of claim 1, wherein the dose of one or a comb nation of herbs is 0.1 g to 1.0 g of dried herb or herbs, preferentially 0.3 g, or its equivalent dried hydro-alcoholic extract.

8. The composition of claim 7, wherein each herb is combined equally.

9. The composition of claim 7, wherein the herbs are administered at least twice daily for at least three days to treat malaria.
